(19) United States
(12) Patent Application Publication
(10) Pub. No.: US 2013/0171703 A1
(43) Pub. Date: Jul. 4, 2013

(54) PRODUCING ENERGY FROM BIOLOGICAL MATERIAL

(71) Applicants: Amnon YOGEV, Rechovot (IL); Eliyahu GAMZON, Moshav Beit Oved (IL)

(72) Inventors: Amnon YOGEV, Rechovot (IL); Eliyahu GAMZON, Moshav Beit Oved (IL)

(73) Assignee: ENGINEUITY RESEARCH AND DEVELOPMENT LTD., Yavne (IL)

(21) Appl. No.: 13/598,934

(22) Filed: Aug. 30, 2012

Related U.S. Application Data
(63) Continuation of application No. 12/668,051, filed on Jan. 7, 2010, now abandoned.
Application No. 12/668,051 is a continuation-in-part of application No. PCT/IL2007/001576, filed on Dec. 19, 2007.
Application No. PCT/IL2007/001576 is a continuation-in-part of application No. 11/818,967, filed on Jul. 13, 2007, now Pat. No. 8,366,966.
(60) Provisional application No. 61/064,043, filed on Feb. 12, 2008.
(60) Provisional application No. 61/064,043, filed on Feb. 12, 2008.

(51) Int. Cl. C12P 3/00; C12P 7/04; C12M 1/107; C12P 7/62

(52) U.S. Cl. CPC C12P 3/00; C12P 7/62; C12P 7/04; C12M 21/04
USPC 435/135; 435/168; 435/157; 435/289.1

(57) ABSTRACT

Described are methods and systems for producing synthesis gas. In some embodiments, the method includes microbiologically converting biological material to form methane and CO2; and reacting methane and CO2 formed in (a) with water and metal to form synthesis gas. Optionally, the method also includes cutting vegetation; fermenting the vegetation to form biogas comprising methane and CO2; and reacting the biogas with water and metal to form synthesis gas and metal oxide. In some embodiments of the invention, reacted metal is regenerated from metal oxide produced in the reaction. In some embodiments the regeneration comprises reacting the oxide in a bath of boiling zinc.

### FIELD OF THE INVENTION

The present invention relates to methods for producing methanol or other fuels from simpler molecules, such as carbon dioxide, water, and/or methane. The invention also relates to apparatuses for carrying out such methods.

### BACKGROUND OF THE INVENTION

In view of the high prices of gasoline, and the environmental problems associated with its wide use, it is desirable to develop an energy carrier that would replace, at least partially, the use of gasoline and reduce CO₂ emission.

One such alternative energy carrier is methanol. A hypothetical future economy based on the idea of using methanol instead of fossil fuels as a means of transportation of energy is sometimes termed “the methanol economy”. The article Beyond Oil and Gas: The Methanol Economy authored by George A. Olah and published in Angewandte Chemie International Edition Volume 44, Issue 18, Pages 2636-2639, 2005, advocates the methanol economy and discusses the generation of methanol from carbon dioxide or methane.

The article “Biogas production from maize and clover grass estimated with the methane energy value system” presented at Engineering the Future, 12-16 Sep. 2004, Leuven, Belgium; and at International Water Association: 10th World Congress—Anaerobic Digestion, Aug. 29-Sep. 2 2004, Montreal Canada discusses biogas production from maize and clover grass with different maize varieties. The article concludes that maize and clover grass silage (that is, green fodder that is stored in a silo and allowed to ferment) are very suitable substrates for anaerobic digestion, and that silaging increases the methane yield from maize and clover grass.

### SUMMARY OF THE INVENTION

An aspect of some embodiments of the invention relates to making fuel that is friendly to the environment, and preferably by consuming greenhouse gases, such as carbon dioxide and methane.

In exemplary embodiments of the invention, the greenhouse gases are taken from microbiological conversion, for example, fermentation, of biological material.

An aspect of some embodiments of the invention relates to making fuel, for instance, methanol, from carbon dioxide (CO₂) and water by reacting them with a metal.

As a by-product, metal oxide is obtained. In preferred embodiments of the invention, the metal is regenerated from the metal oxide. Optionally, the metal is regenerated by electrolysis of the metal oxide. Additionally or alternatively, at least part of the metal is regenerated by reacting the metal oxide with methane, or any other carbonaceous material.

In an exemplary embodiment of the invention, CO₂ is obtained from processes that produce it as a side-product, for instance, in processes that produce methane from biological sources, for instance, maize fermentation.

In another exemplary embodiment of the invention, CO₂ is obtained from sources, where it is naturally present together with methane.

In an embodiment of the invention, CO₂, water, methane and metal are reacted together to produce synthesis gas, also called syngas, and metal oxide. Without being bound to theory, it is assumed that the CO₂ reacts with the metal to produce CO and metal oxide; water reacts with the metal to produce hydrogen and metal oxide, and the methane reacts with the metal oxide to obtain more CO and metal. This way, some of the metal consumed by the water and the CO₂ is regenerated by the methane, and the amount of metal oxide that has to be regenerated by other means, for instance, electricity, is smaller than would be without the methane. In an exemplary embodiment of the invention, presence of methane in the process saves about 90% of the electricity required for regenerating the metal from the metal oxide.

Optionally, a process and/or system according to the invention may be used for storing electrical energy. Many conventional power plants produce at off-peak hours, for instance at night and during week ends, more electrical energy that the consumers consume. There is a need in the art, to store such electrical energy, in a way that allows its use in the peak-usage hours. In an embodiment of the invention, metal is regenerated from the produced metal oxide by electrolysis at off-peak hours, using cheep electricity, and the produced metal is used to produce methanol to replace expensive electricity at peak-usage times. Alternatively or additionally, methanol is produced in off-peak hours, and used in peak hours to produce electricity, for instance, using a gas turbine.

An embodiment of the invention provides an apparatus for producing fuel from biological material, which consumes CO₂ from microbiological conversion of the biological material, water from any available source, and energy. Optionally, energy is obtained from renewable sources, such as solar power, wind energy, hydro energy, or the like. Alternatively or additionally, energy is obtained from conventional carbon based electric power sources. Alternatively or additionally, energy is obtained from combusting other portions of the biological material, for instance, sugarcane straw.

In an exemplary embodiment of the invention, a fuel producing apparatus comprises the following units: I—A fermentation unit—this unit provides CO₂ and methane from biological material, or from any other available source, and releases the CO₂ for use in unit H described below. II—A syngas producing unit—this unit consumes water, the CO₂ and the methane provided by the first unit, and reacts them with a metal to produce hydrogen and carbon monoxide (CO), the mixture thereof is known in the art as synthesis gas, or in short, syngas. This unit produces excess heat and creates a metal oxide as waste. Optionally, the excess heat is used in unit I, and the waste metal oxide is turned into metal in unit IV.

III—A fuel producing unit this unit produces carbonaceous fuel, for instance, methanol, from the syngas produced by the syngas producing unit. IV—an optional metal regenerating unit, for regenerating the metal from the metal oxide produced in the syngas producing unit. The metal recycling unit may reduce considerably the metal consumption of the entire apparatus, and in some cases reduce this consumption to zero.

Optionally, the metal recycling unit comprises an electrolysis bath, configured to electrolyze the metal oxide to produce the metal.

In exemplary embodiments of the invention, hydrogen produced by the electrolysis of the metal oxide is added to the fuel producing unit, thus saving at least a portion of the metal and the electricity required for recycling this portion of metal. Other hydrogen sources may also be used for supplying hydrogen to the fuel producing unit, in addition, or instead of, reacting water with the metal.

Alternatively or additionally, the metal recycling unit comprises a reaction chamber for reacting the metal oxide with methane or other low grade carbonaceous material as to regenerate the metal. Optionally, such metal recycling unit receives heat from a heat source, for instance, a solar heat source, and/or heat from the syngas producing unit.

In an exemplary embodiment, the metal is zinc, and it is regenerated from the zinc oxide by endothermically reacting the oxide with carbon. This endothermic reaction produces CO and zinc. Optionally, the zinc oxide and the carbon are reacted with each other in boiling zinc.

Optionally, the CO obtained in the reaction is oxidized with air to produce heat, and this heat is used to feed the endothermic reaction between zinc oxide and carbon. Optionally, the amount of zinc leaving the reactor by evaporation (due to boiling) is balanced with the amount of zinc produced in the reactor from zinc oxide.

Optionally, carbon is produced in situ by heating carbonaceous material, for instance, sawdust, to produce carbon and gases, and these gases are oxidized to provide the heat required for the endothermic reaction. Preferably, heating is in the absence of air, as to result in pyrolysis. Optionally, the heating is with some air, sufficient to partially oxidize the carbonaceous material.

in another exemplary embodiment, the zinc oxide is reacted with carbonaceous material to produce zinc and combustible gases. These gases are optionally used similarly to CO in the preceding example.

Alternatively or additionally, metal recycling may be accomplished by heating the metal oxide to sufficiently high temperatures. Heat for such recycling process may be obtained, for instance, from solar energy. Optionally, such heating is in the presence of carbonaceous material, such as methane, or vegetation. In the presence of carbonaceous material less heating may be required than in the absence thereof.

Electric power may be supplied to the various units I-IV from any available power source, such as national or regional power plant. Alternatively or additionally, the apparatus includes a fifth unit (hereinafter unit V), which produces electric power for operating the other units from locally available renewable sources such as wind or solar energy. Optionally, unit V utilizes combustion of a biological material to produce the electric power.

Thus, an aspect of some embodiments of the invention concerns a method of producing synthesis gas, the method comprising:

(a) microbiologically converting biological material to form methane and CO2; and

(b) reacting methane and CO2 formed in (a) with water and metal to form synthesis gas.

In some exemplary embodiments, the method further comprises cutting vegetation; fermenting the vegetation to form biogas comprising methane and CO2; and reacting the biogas with water and metal to form synthesis gas and metal oxide.

In an exemplary embodiment, the method comprises silaging the cut vegetation, and fermenting silaged vegetation.

Optionally, silaging comprises obtaining compost.

In some exemplary embodiments of the invention, the method further comprises recycling the metal oxide to obtain metal.

In an exemplary embodiment, the metal is zinc; and recycling the metal oxide comprises reacting the zinc oxide with carbonaceous material in boiling zinc.

Optionally, such method comprises condensing zinc vapor of the boiling zinc to obtain liquid zinc; and reacting methane and CO2 formed in in the biological conversion with the obtained liquid zinc.

Optionally, the carbonaceous material is carbon.

Optionally, the method comprises obtaining the carbon in a method comprising gasifying biomass to obtain carbon and combustible gases. Optionally, the method further comprises oxidizing the obtained combustible gases; and heating the boiling zinc with heat obtained from oxidizing the combustible gases. The heating is optionally from outside, for instance, with a heat exchanger.

In some embodiments of the invention, recycling comprises electrolyzing. In some embodiments of the invention, recycling comprises thermally decomposing. Optionally, thermally decomposing comprises heating with solar thermal energy. Optionally, thermally decomposing is in the presence of carbonaceous material.

Optionally, the carbonaceous material comprises at least one of vegetation and methane. Optionally, the vegetation comprises one or more of switchgrass (Panicum virgatum), hay, forage, sugarcane or portion thereof, and starch forming plant, such as maize, rice, wheat, barley, sorghum, oats, millets, rye, triticale, and buckwheat, in some embodiments, recycling comprises utilizing electrical power, and the method comprises combusting portions of the vegetation to produce at least a portion of said electrical power.

In exemplary embodiments of the invention, the biological material microbiologically converted in (a) comprises at least one of switchgrass (Panicum virgatum), hay, and forage.

In an exemplary embodiment, the biological material comprises sugarcane. In an exemplary embodiment, the biological material comprises sugarcane bagasse or portions of sugarcane that are not fermentable to ethanol.

In some exemplary embodiments, the biological material comprises green starch forming plant.

Optionally, the green starch forming plant is not fully ripe. Optionally, the green starch forming plant is not ripe enough to be harvested for ethanol production.

Optionally, the green starch forming plant is not ripe enough to be suitable as foods for humans. Optionally, the green starch forming plant is not ripe enough to be suitable as food for livestock.

In some exemplary embodiments, the green starch forming plants comprises at least one of maize, rice, wheat, barley, sorghum, oats, millets, rye, triticale, and buckwheat.

In an exemplary embodiment, the method also comprises (d) combusting a portion of the biological material to obtain electricity.

Preferably, in such embodiment recycling the metal oxide comprises utilizing the electricity obtained in (d).

In an exemplary embodiment, the biological material comprises sugarcane, the sugarcane comprises sugar, bagasse, and leaves and stems, and the method comprises:

(a1) microbiologically converting the bagasse to form biogas comprising methane and CO2;

(b1) reacting biogas formed in (a) with water and metal to form synthesis gas and metal oxide;

(c1) combusting sugarcane leaves and/or stems to form electric power; and

(d1) electrolyzing the metal oxide to regenerate the metal, said electrolyzing comprises utilizing electric power formed in (c1).

Another aspect of some embodiments of the invention concerns a method of producing methanol, the method comprising: producing synthesis gas in a method according to any embodiment of the invention and processing the synthesis gas to form methanol.

Another aspect of some embodiments concerns a method of forming an ester, the method comprising; forming alcohol from a first vegetation in a process comprising microbilological conversion of the vegetation; microbiologically converting a second vegetation to form a carboxylic acid; and reacting said alcohol with said carboxylic acid to obtain the ester. In some exemplary embodiments, the process comprises: microbiologically converting the second vegetation to biogas comprising CO2 and methane; reacting: the biogas with water and metal to obtain syngas and metal oxide; and producing an alcohol from the syngas. Optionally, the alcohol comprises methanol. Optionally, the carboxylic acid comprises at least one of succinic acid and butyric acid. Optionally, the process comprises biologically converting the second vegetation to ethanol.

Optionally, the first vegetation is sugarcane bagasse; and the second vegetation is sugarcane sugar.

Optionally, the method comprises recycling the metal oxide to from the metal. Optionally, recycling comprises electrolyzing. Alternatively or additionally, recycling comprises utilizing electrical energy. In some preferred embodiments, the method comprises forming the electrical energy in a process comprising combusting vegetation.

δ

Optionally, recycling comprises thermally decomposing in the presence of carbonaceous material. Optionally, heating is with solar thermal energy. Optionally, thermally decomposing is in the presence of carbonaceous material.

Another aspect of some embodiments of the invention concerns system for producing liquid fuel comprising:

(a) a fermentation unit, having an inlet for biological material and an outlet for biogas,

(b) a syngas producing unit, configured for receiving said biogas and water, and reacting said biogas with a first metal and said water with a second metal, which is the same or different from said first metal, to produce hydrogen and carbon monoxide;

(c) a fuel producing unit, configured for producing a carbonaceous fuel from products of the syngas producing unit; and

(d) a metal regenerating unit, configured for regenerating metal from metal oxide produced in the syngas producing unit.

In an exemplary embodiment, the metal regenerating unit comprises: a carbon source; a reactor with liquid zinc, having an inlet for receiving metal oxide from the syngas producing unit, an inlet for receiving carbonaceous material from said carbon source, and an outlet for exiting gaseous zinc,

Optionally, the metal regenerating unit also comprises a heat exchanger, keeping the temperature of said reactor above the boiling temperature of zinc.

Optionally, the heat exchanger receives heat from a combustor, which receives combustible gas from said reactor and combusts the received combustible gas.

In some exemplary embodiments, the inlet receives carbon from a pyrolysis chamber, wherein carbonaceous material is pyrolized. Optionally, the carbonaceous material is pyrolized in the absence of air. Optionally the carbonaceous material comprises vegetation, switchgrass (Panicum virgatum), hay, forage, and waste residues from processing plant materials, for instance, sugarcane bagasse vinasse, and/or sawdust. Optionally, the carbonaceous material comprises carbon. Optionally, the carbonaceous material. In some embodiments of the invention, the system comprises an electric power producing unit, configured to supply electric power to said metal regenerating unit.

Optionally, the electric power producing unit is configured to utilize renewable energy sources.

Optionally, the electric power producing unit comprising a combustion chamber for combusting biological material.

In some exemplary embodiments, the syngas producing unit comprises:

(a) a container with liquid metal,

(b) a reaction chamber, configured for receiving liquid metal from said container and comprises: an inlet for receiving biogas, and an outlet for letting out syngas produced in the reaction chamber; and

(c) a heat transferring member for transferring heat from said reaction chamber to said container.

Optionally, the container with liquid metal has a metal inlet, for introducing metal in solid state into said container. Optionally, the inlet comprises elastic seals.

In some embodiments, the heat transferring: member is a portion of the container, and this portion directly contacts liquid metal in the container.

In some embodiments of the invention, the reaction chamber and said container are concentric. In some embodiments of the invention, the metal regenerating unit comprises an electrolytic bath.

In some exemplary embodiments of the invention, the reaction chamber is configured for receiving a carbonaceous material and reacting the carbonaceous material with a metal oxide produced by a reaction between the metal and the biogas and water. Optionally, the carbonaceous material is methane.

In some embodiments of the invention, the first and second metals are the same.

In some exemplary embodiments of the invention, the metal regeneration unit comprises: a conduit, for receiving metal oxide from said syngas producing unit and transferring said metal oxide to a metal reproducing unit; a heat exchanger, for cooling said metal oxide; a first valve and a second valve defining between them along said conduit an intermediate zone, said first valve connecting said intermediate zone to said syngas producing unit and said second valve connecting said intermediate zone to said metal reproducing unit.

Optionally, such a system comprises acid inlet for introducing an acid into said intermediate zone.

Optionally, the metal reproducing unit comprises an electrolytic bath. Optionally, the reproducing unit comprises a solar heater, capable of heating said metal oxide as to decompose it.

Optionally, the reproducing unit comprises an inlet for a carbonaceous material.

An aspect of some embodiments of the invention concerns a system for producing liquid fuel, the system comprising:

(a) a bagasse fermentation unit, having an outlet for biogas, the bagasse fermentation unit being configured for producing biogas from at least one of bagasse and other sugarcane portions not fermentable to ethanol, such as vinasse.

(b) a syngas producing unit, configured for receiving biogas from the bagasse fermentation unit, and for producing syngas from said biogas;

(c) a fuel producing unit, configured for producing a carbonaceous fuel from products of the syngas producing unit; and

(d) a metal regenerating unit, configured for regenerating metal from metal oxide produced in the syngas producing unit.

In some exemplary embodiments, the metal regenerating unit comprises; a carbon source; a reactor with liquid zinc, having an inlet for receiving metal oxide from the syngas producing unit, an inlet for receiving carbonaceous material from said carbon source, and an outlet for exiting gaseous zinc. In an exemplary embodiment, the metal regenerating unit also comprises a heat exchanger, keeping the temperature of said reactor above the boiling temperature of zinc.

Optionally, the heat exchanger receives heat from a combustor, which receives combustible gas from said reactor and combusts the received combustible gas.

In an exemplary embodiment, the carbon source comprises a pyrolysis chamber for pyrolyzing carbonaceous material.

Optionally, the pyrolysis chamber is configured for pyrloyzing the carbonaceous material in the absence of air.

Optionally, the carbonaceous material received in the reactor from said carbon source is carbon. Alternatively or additionally, the carbonaceous material comprises vegetation, switchgrass (Panicum virgatum), hay, forage, and waste residues from processing plant materials, for instance, sugarcane bagasse vinasse, and/or sawdust. In an exemplary embodiment of the invention, the system further comprises a sugar fermentation unit, configured to produce ethanol from sugar. In an exemplary embodiment of the invention, the system comprises an electric power producing unit, configured to supply electric power to said metal regenerating unit.

Optionally, the electric power producing unit comprises a combustion chamber configured for combusting sugarcane leaves and stems.

Another aspect of some embodiments of the invention concerns a method of producing synthesis gas from sugarcane comprising sugar, bagasse, and leaves and stems, the method comprising:

(a) microbiologically converting sugarcane bagasse to form methane and CO2; (b) reacting methane and CO2 formed in (a) with water and metal to form synthesis gas;

(c) combusting sugarcane leaves and/or stems to form electric power; and

(d) electrolyzing the metal oxide to regenerate the metal, said electrolyzing comprises utilizing electric power formed in (c).

### BRIEF DESCRIPTION OF THE DRAWINGS

Embodiments of the invention are herein described, by way of example only, with reference to the accompanying drawings. It is stressed that the particulars shown are for purposes of illustrative discussion of the described embodiments, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how several forms of the invention may be embodied in practice. In the drawings:

FIG. 1A is a simplified block diagram of a system for producing syngas according to an embodiment of the invention;

FIG. 1B is a schematic illustration of powder inlet in accordance with an embodiment of the present invention;

FIGS. 2A and 2B are schematic illustrations of syngas producing units according to embodiments of the invention;

FIG. 3A is a schematic illustration of an oxide removing device, for removing oxide from a syngas producing unit according to an embodiment of the invention;

FIG. 3B is a schematic illustration of a zinc regenerating unit according to an embodiment of the invention; and FIG. 4 is a flowchart describing actions taken in a method of producing ethanol and methanol from sugarcane according to an embodiment of the invention.

### DETAILED DESCRIPTION OF SOME EMBODIMENTS OF THE INVENTION

In the following, some general features of exemplary embodiments of the invention are first outlined, and then, specific embodiments are described in detail.

Microbiologically Converting Biological Material

Microbiologically converting the biological material is optionally carried out in known methods. One of many such methods may be found in U.S. Pat. No. 6,454,944 to Raven, titled “Process and apparatus for conversion of biodegradable organic materials into product gas”.

Conditions for Producing Syngas from Water, Carbon Dioxide, and Methane

In some embodiments of the invention, methane or any other carbonaceous material is also introduced into the syngas producing unit. In such embodiments, it is optional to operate the unit with carbon dioxide:methane:water molar ratios of 1:1:2, which leaves no excess of either of the reactants. Here also, preferable working temperature is around 800° C., and excess of carbon sources over the hydrogen source, allows working at a broader temperature range. At higher pressure, higher temperature is required.

As the reaction between methane and metal oxide is endothermic, in some embodiments it consumes the heat produced by the exothermic reactions of water and/or carbon dioxide with the metal, and lowers the temperature in the syngas producing: unit to below preferable operation temperature. In these embodiments, it may be advisable to supply the syngas producing unit with heat from an external source, for example, from the fuel-producing unit.

It should be noted that the amount of energy required for recycling the metal per product unit depends on the relative amount of methane used.

For example, in the absence of methane, about 10 kWh electricity is required to regenerate by electrolysis metal used for producing 1 kg of methanol.

If methane is used in a quantity sufficient to carry out the reaction (I), electrical energy required for recycling is only about 3.3 kWh/kg methanol. (I) CO_{2(g)}+CH₄(g)+2H₂O+2ZnO=2CH₃OH+2ZnO

If methane is used in a quantity sufficient to carry out the reaction (II), electrical energy required for recycling is only about 1 kWh/kg methanol.

CO_{2(g)}+2CH_{4(g)}+2H₂O+Zn=3CH₃OH+ZnO (II)

In some embodiments, the regeneration comprises reacting the metal oxide with carbon, optionally solid carbon. In such embodiments, using more methane in the syngas formation reaction permits using less carbon in the metal regeneration process.

Syngas Producing Reactions

Preferably, metal is introduced into the reaction chamber in small droplets, to let the gases react with the metal faster. Introducing the metal as droplets may be with any commercially available metal sprayer, as, for instance, those used for spraying corrosion-resistant coatings, such as zinc or aluminum.

The metals are preferably but not limited to Al, Mg, and Zn, or alloys of these metals or alloys of one or more of these metals with other metals.

In some embodiments, metal oxide produced in the reaction chamber sinks on the floor of the reaction chamber due to its relatively high density. This metal oxide is optionally removed from the reaction chamber through an outlet in the floor of the reaction chamber.

Optionally, the removed metal oxide is then cooled, and reacted with an acid to provide an aqueous solution. Optionally, the produced aqueous solution is electrolyzed to obtain metal that is optionally used again to react with fresh water and CO₂.

In some embodiments, part or all of the metal oxide is created as a powder made of very small particles, and rather than sinking to the floor of the reaction chamber, this powder floats and creates together with the produced gas an aerosol. In this embodiment, the metal oxide is removed from the reaction chamber together with the produced gases. In an embodiment of the invention, the produced aerosol is cooled, and bubbled into an electrolysis solution, in which the metal oxide reacts to produce metal and the gases contained in the aerosol leave the electrolysis solution free of metal oxide powder.

Heat Economy

In a preferred embodiment of the invention, heat produced in one or more of the exothermic (heat producing) processes is supplied to the endothermic (heat consuming) processes.

The heat endothermic processes optionally include reacting carbonaceous material with metal oxide, and/or regenerating the metal and the exothermic processes optionally include;

Reacting a metal with water, CO₂ and/or methane; •Cooling the metal oxide before hydrolyzing it; and

Cooling the syngas and producing fuel therefrom.

Optionally, heat produced in one or more of the exothermic processes is used to operate a heat machine, for instance, a turbine.

In an exemplary embodiment of the invention, syngas exits the syngas producing unit at about 800° is cooled or heated to a temperature selected for producing the fuel in the fuel producing unit.

Optionally, syngas exits the syngas producing unit at a pressure suitable for producing the fuel at the selected temperature.

Optionally, cooling the syngas comprises expanding it through a gas turbine, thus utilizing the heat carried by the syngas to produce work. Additionally or alternatively, heat excess from any of the units in a system according to embodiments of the invention may be used for operating one or more steam turbine(s).

It is also possible to use excess heat for heating houses, water in domestic water systems, etc.

Sources of CO₂

CO₂ is optionally supplied with gas mixtures that are rich with CO₂. One example of such a gas mixture is natural gas. In many wells of natural gas, CO₂ is present together with the natural gas (methane). Usually, the CO₂ is treated as a contamination, separated from the methane, and released to the atmosphere. In accordance with an embodiment of the present invention, rather than separating CO₂ from methane, the two gases are reacted together, in the presence of water and a metal, to form synthesis gas, which is preferably used for producing a liquid fuel, such as methanol. The liquid fuel is advantageous over the natural gas in that it may be easily transferred; it does not leak as easily as gas does; it is biodegradable, and is less dangerous in handling.

Similarly, methane and carbon dioxide are produced from solid waste, for instance, from landfills, but usually only the methane is used. In accordance with an embodiment of the present invention, the carbon dioxide is also used, to produce with the methane a liquid hydrocarbon, such as methanol.

Fermentation facilities, used for producing ethanol as a gasoline substitute, are another optional CO₂ source. Fermentation facilities produce ethanol from fermentation of biological material. In most cases, the fermentation product includes CO₂ that is not used in the formation of ethanol. Using embodiments of the present invention to form methanol from this CO₂ may double the energy produced by these facilities, and save the environment from considerable amounts of CO₂.

Many other known processes produce carbon dioxide as a by-product, and release it to air, thus exacerbating the greenhouse effect. It is in accordance with embodiments of the present invention to use carbon dioxide from any such process, for forming syngas, and optionally, from the syngas, a hydrocarbon based fuel, preferably a liquid fuel.

In an embodiment of the invention, syngas is produced from CO₂ and methane obtained from microbiological conversion of biological materials or waste, for example, vegetation, switchgrass (Panicum virgatum), hay, forage, and waste residues from processing plant materials, for instance, sugarcane bagasse and/or vinasse.

In one embodiment, the vegetation comprises starch foaming plants, for example, maize, rice, wheat, barley, sorghum, oats, millets, rye, triticale, and/or buckwheat.

Optionally, the vegetation is harvested green, since at this stage it contains more cellulose and less starch than in later stages. Cellulose is richer in energy than lignin or starch. Optionally, starch forming plant is considered green if it is not fully ripe, that is, if starch formation is not complete. Alternatively, starch forming plant is considered green if it is not ripe enough to be harvested for ethanol production.

Alternatively, starch forming plant is considered green if it is no ripe enough to be suitable as foods for humans. Still alternatively, starch forming plant is considered green if it is no ripe enough to be suitable as food for livestock.

For example, maize is a starch forming plant, which if harvested before ripening of the grains to complete starch formation, may produce three times more energy than if harvested when the grains are ripe. In some embodiments of the invention, syngas is produced from CO₂ and methane obtained from microbiological conversion of bagasse, which is the biomass remaining after sugarcane stalks are crushed to extract their juice. In some embodiments of the invention, syngas is produced from CO₂ and methane obtained from microbiological conversion of sugar juice content which is not fermented to ethanol. Optionally, sugar juice content which not fermented to ethanol comprises vinasse.

Optionally, vegetation portions that are less useful for producing methane and/or CO₂, are used as fuel for thermal processes, to provide electric power. In an embodiment of the invention, electric power obtained this way is used for running the syngas production. For instance, if the metal is regenerated by electrolysis, the electricity may be used for metal regeneration, and the system may be self-contained.

Examples of vegetation portions that are optionally used as fuel for thermal processes include straw and leaves.

In an embodiment of the invention, non-edible portions of plants are biologically converted to biogas, while edible portions are used as food. This embodiment may allow utilizing the vegetation for both food and fuel production.

In an embodiment of the invention, vegetation is harvested green, thus allowing shorter life cycle of the crops, and growing more crops per hectare per year.

In some embodiments of the invention, the various portions of sugarcane: sugar, bagasse, and straw, are processed to obtain liquid fuel.

In one embodiment, the liquid fuel comprises methanol and ethanol. Ethanol is optionally produced in known methods from the sugar juice. Methanol is optionally produced from portions of sugar juice that are not fermented to ethanol, comprising what is referred to in the art as vinasse. In this option, the vinasse is fermented to provide biogas containing carbon dioxide and methane, and the biogas is reacted with water and metal to provide synthesis gas, which in turn may be made into methanol. Additionally or alternatively, the biogas may be obtained from bagasse fermentation.

In another embodiment, the liquid fuel comprises esters of carboxylic acids. Optionally, such esters may be used as fuel for diesel engines.

A method according to this embodiment comprises: fermenting vegetation to obtain carboxylic acid; forming an alcohol in a process comprising fermenting vegetation; and reacting the alcohol and the carboxylic acid to obtain ester.

Optionally, the process of forming an alcohol comprises fermenting the vegetation to biogas, and reacting the biogas to form alcohol.

Optionally, the vegetation fermented to alcohol and the vegetation fermented to biogas are of the same kind. Optionally, these are two portions of the same plants, for instance, stems and fruits. Optionally, these are two different plants.

One such method, where the vegetation fermented to alcohol and that fermented to biogas are two portions of the same plant comprises, for example: fermenting sugar to obtain a carboxylic acid; processing bagasse, to obtain biogas comprising methane and carbon dioxide; processing the biogas to obtain alcohol: and reacting the obtained alcohol with the obtained carboxylic acid.

Optionally, processing the biogas to obtain alcohol comprises reacting the biogas with water and metal. Reacting the alcohol with the acid is optionally carried out in a method known in the art.

Optionally, the carboxylic acid is succinic acid, butyric acid, or a mixture thereof. Other aliphatic or aromatic carboxylic acids may also be formed from the sugar and reacted with the methanol according to embodiments of the invention.

Optionally, succinic acid is produced using BSDA technology, commercially available from Pacific Northwest National Laboratory (WA, USA).

Energy Sources

Some of the embodiments of the present invention consume energy. Optionally, this energy is obtained from renewable sources, such as solar energy, nuclear energy, hydroelectric energy, geothermic energy, and/or wind.

Additionally, heat produced in some steps of the inventive processes may be used to replace external energy source.

Some embodiments of the invention comprise producing metal from metal oxide. This is optionally done by electrolysis at night, when electricity is relatively cheap.

Exemplary Syngas Producing Apparatus

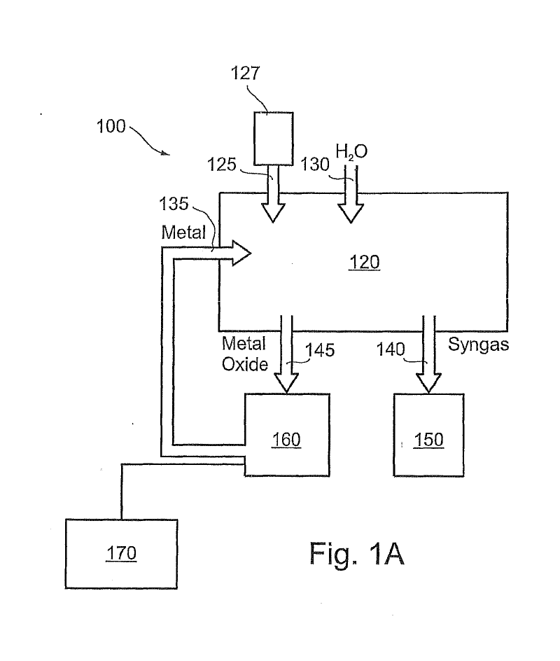

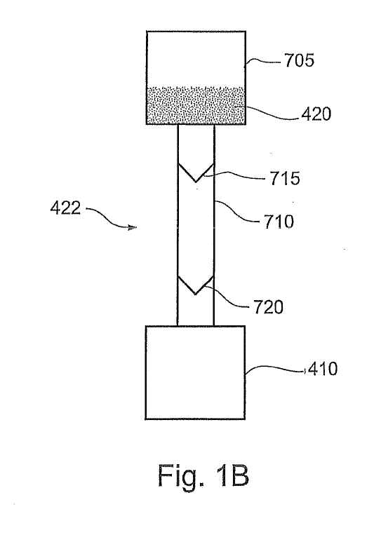

Reference is now made to the figures, which together with the above descriptions, illustrate preferred embodiments thereof. FIG. 1A is a simplified block diagram of an apparatus 100 for producing syngas from biological material and water according to an embodiment of the invention. In the figure, block 120 is a syngas producing unit. Syngas producing unit 120 has an inlet 125, for receiving CO₂ and methane. Optionally, CO₂ and methane are received from a fermentation unit 127. Fermentation unit 127 is of a kind known in the art per se. Optionally, inlet 125 also receives water vapor. Optionally, syngas producing unit 120 has a separate water inlet 130, but this may be omitted if the CO₂ is supplied with sufficient amounts of water. Optionally, syngas producing unit 120 has only one inlet, for receiving all the non-metallic reactants.

In embodiments where the CO₂ enters syngas producing unit 120 free of water, unit 120 may include two reaction chambers (not shown), one for reacting the CO₂ with a first metal, and the other for reacting water with a second metal, which may be the same or different from the first metal. In other cases, the CO₂ and the water may react in a single reaction chamber. Optionally, metal is introduced into the syngas producing, unit through a metal inlet, 135.

Syngas producing unit 120 comprises a syngas outlet 140. Optionally, the syngas leaves unit 120 at a pressure, which is most suitable for producing methanol, that is, at about 50 Atm, although other pressures may he used. In some embodiments of the invention, the temperature and/or pressure is adjusted to the temperature and/or pressure required in fuel producing unit 150 after the syngas leaves unit 120.

Optionally, syngas outlet 140 supplies the syngas to a fuel producing unit 150, which produces fuel, for instance, methanol from the syngas by any method known in the art per se. Production of methanol from syngas is described, for instance in Kirk Othmer Encyclopedia of Chemical Technology published by Wiley Interscience.

Syngas unit 120 also comprises a metal oxide outlet 145, leading into a metal regeneration unit, 160.

Optionally, metal regeneration unit 160 comprises an electrolysis bath, in which the metal oxide is electrolyzed to produce a metal. Optionally, the electrolysis bath receives electrical power from power source 170. Power source 170 is optionally a national or regional power plant, or a local power source. A local power source optionally uses combustion of biological material to produce electric power.

Additionally or alternatively, local power source may produce power from renewable sources, such as solar energy, wind, hydroelectric power, etc.

Optionally, metal produced in regeneration unit 160 is introduced to the syngas producing, unit, for instance, through inlet 135.

In a preferred embodiment of the invention, syngas producing unit 120 is independent of metal regeneration unit 160, such that syngas is produced irrespective of metal regeneration. This allows regenerating the metal only when cheep electricity supply is available, while producing syngas around the clock. Metal regeneration or fuel production may be limited, for instance, to daytime, when solar energy is available, to night time, when central power plants provide cheap electric power, or whenever there is wind that is capable of operating a wind driven power plant, all in accordance with the specific kind of power source 170 that is being used.

In operation, water reacts with a metal to form hydrogen and metal oxide and CO₂ reacts with a metal to form CO and metal oxide. In exemplary embodiments of the invention, these reactions may require ignition. Ignition is optionally supplied by electric spark, discharge, and/or a hot filament.

Metal is preferably introduced into syngas producing unit 120 during operation. Metal introduction is optionally synchronous with introduction of the other reactants, such that there is no excess of metal over the other reactants. Excess of metal may turn the reaction in the opposite direction, to produce CO₂ from CO and metal oxide. Metal is optionally introduced into the syngas producing: unit by sprinkling with a metal sprayer, for instance, of the kind used for spraying metals to coat substrates. Syngas produced by the reactions between CO₂, water, and metal in the syngas producing unit (120) exits into methanol producing unit 150, wherein fuel is catalytically prepared from the syngas using methods that are known in the art. Optionally, the fuel is an alcohol, such as methanol or ethanol, or any other hydrocarbon. Metal oxide produced by the reactions that take place in the syngas producing unit exits into metal regeneration unit, 160, where it is reacted to regenerate the metal. The regenerated metal is optionally returned to syngas producing unit 120, to react with fresh amounts of water and CO₂.

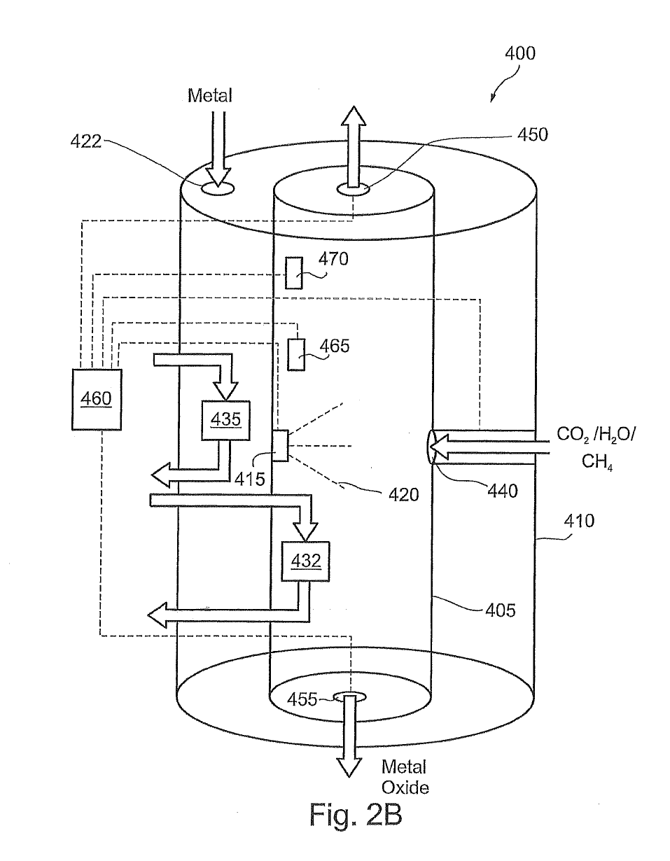

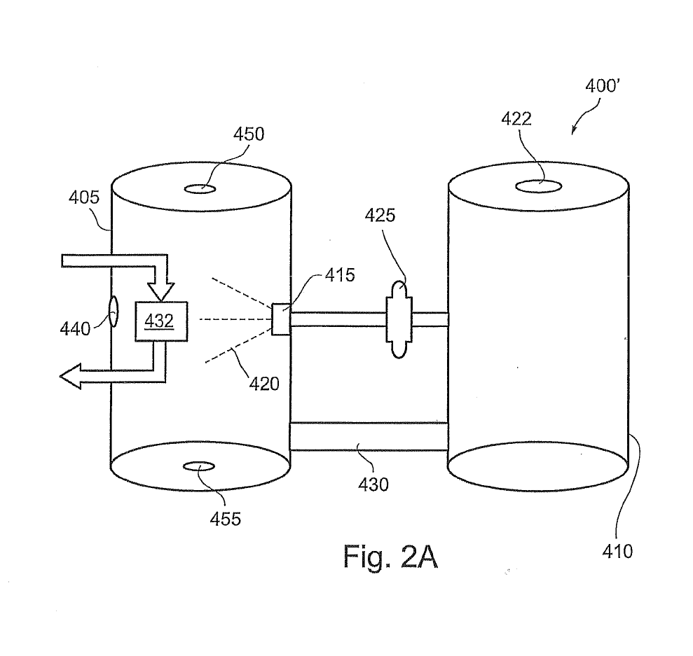

FIGS. 2A and 2B are simplified schematic illustrations of syngas producing units 400 and 400′ for oxidizing metals with water to form hydrogen and metal oxide and/or for oxidizing metals with CO₂ to form CO and metal oxide. Another suitable syngas producing unit is described in Applicants' patent application WO2006/123330, the disclosure of which is incorporated by reference. Other methods, known in the art for producing syngas from CO₂ and water can also be used, in some embodiments of the invention.

Devices 400 and 400′ comprise two containers. One container (405) is a reaction chamber, and the other container (410) contains the metal (420) in liquid form.

Reaction chamber 405 comprises a sprinkle or nozzle (415), configured to supply liquid metal 420 from container 410 to the reaction chamber.

Optionally, metal 420 is supplied to container 410 in solid state, for instance, in the form of powder, a rod or a wire, and heated to liquefy and/or to remain at liquid state by heat produced in reaction chamber 405 during operation.

In an embodiment where metal 420 is supplied as a rod or wire, pressure is optionally used to force the metal into the container through inlet 422. Optionally, inlet 422 comprises elastic seals, as described, for instance, in Applicants' patent application No. WO2006/123330, incorporated herein by reference.

Exemplary Powder-Fed Syngas Producing Unit

In an embodiment of the invention, metal 420 is supplied as powder. Optionally, in such an embodiment, inlet 422 is as described in FIG. 1B.

In FIG. 1B, inlet 422 is shown to be in communication with a powder reservoir 705 having metal powder 420 through a powder conduit 710 having two valves, 715 and 720. Valve 715 separates conduit 710 from reservoir 705, and valve 720 separates conduit 710 from container 410. In operation, when valve 720 is closed, valve 715 is opened and allows introducing metal powder into conduit 710. Then, valve 715 is closed, and valve 710 is opened to transfer the powder to container 410.

Optionally, the pressure used to force the metal into container 405 is utilized for pushing liquid metal 420 into reaction chamber 405. Additionally or alternatively, a pump 425 is used for said pushing.

Exemplary Heat Transfer Arrangements

In FIG. 2A, a heat exchanger 430 is used to transfer the heat from reaction chamber 405 to container 410.

In FIG. 2B, the two containers 405 and 410 are structured in a concentric structure, allowing the absence of a heat exchanger between them.

Optionally, the reaction chamber 405 has in it a heat exchanger 432 for cooling the atmosphere in the reaction chamber, to allow evacuating heat from the reaction chamber as to push forward the exothermic reactions that take place in the reaction chamber.

An external heat exchanger 435 is optionally used for supplying heat from reaction chamber 405 (or metal container 41(B to other applications, for instance, for a CO₂ separation unit.

Reaction chamber 405 is equipped with at least one inlet 440, configured to inlet at least one of water, carbon dioxide, and methane. Optionally, reaction chamber 405 has one, two, or more additional inlets, each configured to inlet at least one of water, carbon dioxide and methane.

In an exemplary embodiment of the invention, inlet 440 is for inletting methane and carbon dioxide together, optionally, from a source having them together, for instance, a fermentation unit.

In an exemplary embodiment of the invention, inlet 440 is adopted for inletting water carbon dioxide, and methane together.

Optionally, the pressure in reaction chamber 405 is designed to fit the pressure under which the syngas has to react in the fuel producing unit to give fuel. For instance, if methanol is to be produced at about 50 Atm (5000 kPa), the syngas producing unit is optionally operated at 50 Atm.

Optionally, inlet 440 is configured for simultaneous inletting of water, CO₂ and methane.

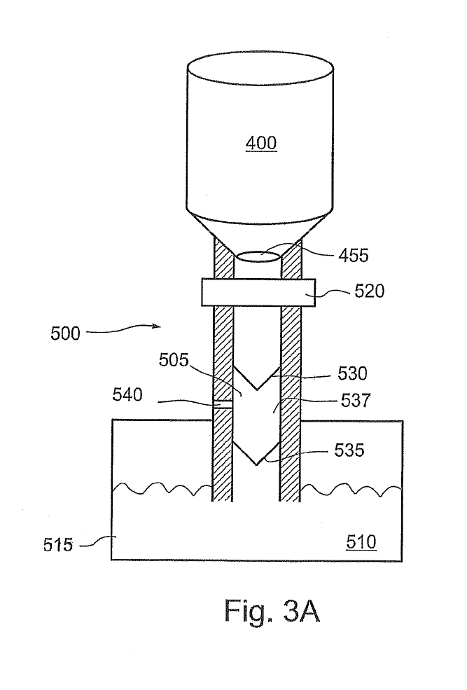

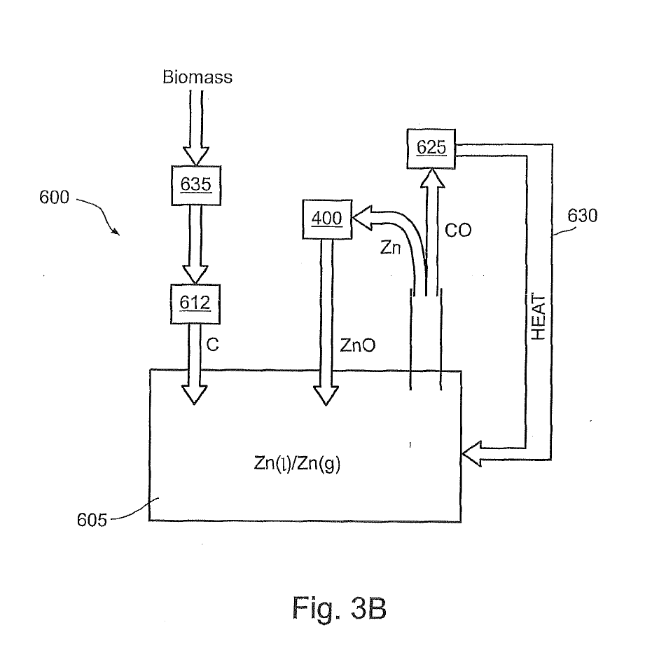

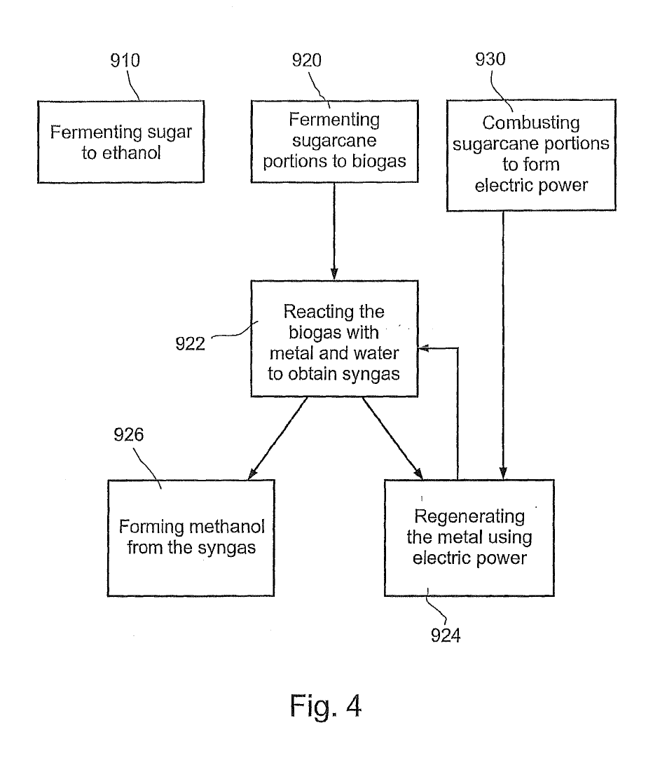

Reaction chamber 405 also has a syngas outlet 450 and optionally a metal oxide outlet 455. An oxide outlet according to an exemplary embodiment of the invention is described bellow in relation to FIG. 3A. In some embodiments, the metal oxide exits together with the syngas, and a separate outlet such as 455 is optionally omitted.

A control system (460) controls the syngas composition by controlling the rate of introduction of the reactants (including metal, and at least one of water, carbon dioxide, and methane) into the reaction chamber (405). Control system 460 optionally also controls syngas outlet 450, and/or oxide outlet 455.

Additionally or alternatively, syngas outlet 450 is controlled by a fuel producing unit (for example unit 150 in FIG. 1) that receives the syngas leaving from the outlet. Optionally, the fuel producing unit communicates with the syngas producing unit through control system 460. Optionally, control system 460 receives data on temperature and pressure inside reaction chamber 405 from temperature sensor 465 and pressure sensor 470. Preferably, device 400 or 400′ comprises a plurality of temperature sensors, for sensing temperature at a plurality of locations inside reaction chamber 405.

Ignition of the metal in reaction chamber 405 is obtained, for example, by electric spark, discharge, or a hot filament.

Exemplary Metal Regeneration

FIG. 3A is a schematic illustration of an oxide removing device 500, for removing oxide from a syngas producing unit 400 according to an embodiment of the invention. Details of unit 400 are not provided, except for outlet 455. Optionally, unit 400 has a conical lower surface with the tip being at outlet 455, such that oxide particles formed during operation of unit 400 concentrate by gravity at outlet 455. Smaller or lighter oxide particles, that do not fall down, but rather go up with the syngas stream are not treated by device 500.

Device 500 comprises a conduit 505, optionally leading into an electrolytic bath 510. Conduit 505 has an upper gate valve 530 and a lower gate valve 535, defining between them an intermediate zone (537). An acid inlet 540 is provided for introducing acid into intermediate zone 537. A heat exchanger 520 is positioned as to cool particles going from unit 400 towards conduit 505.

In operation, oxide particles coming into conduit 505 from syngas producing unit 400 are first cooled by heat exchanger 520, and then enter the conduit through upper gate valve 530. Acid, for example sulfuric acid, is entered into conduit 505 through acid inlet 540 to dissolve the oxide particles. Lower gate valve 535 opens to allow the metal oxide particles dissolved in the acid to pour into bath 510. Optionally, the metal oxide is electrolyzed in bath 510. The pressure in the syngas producing unit 400 is preferably higher than the pressure in the conduit (the latter being optionally atmospheric pressure), to facilitate movement of oxide particles down into conduit 505 and prevent movement of acid up through the conduit.

In an embodiment of the invention, where metal regeneration is not by electrolysis, the use of acid to dissolve the metal oxide and/or the use of an electrolyzer may be omitted. In these embodiments the metal oxide is optionally cooled, and transferred for regeneration by reaction with methane, by heat, or by any other way known in the art per se.

FIG. 3B is a block diagram of a zinc regenerating unit 600 according to an embodiment of the invention.

Unit 600 comprises a reactor 605, filled with boiling zinc. Unit 600 receives zinc oxide particles from syngas producing unit 400, and carbon particles from carbon reservoir 612. Both carbon and zinc oxide are less dense than liquid zinc, and therefore, in some embodiments, they float to the outer surface of bubbles of the boiling zinc, so as to mix the zinc.

The zinc oxide particles react with the carbon particles to produce zinc and CO. Gaseous CO and gaseous zinc exit from reactor 605. The gaseous zinc liquefies upon cooling to below the melting temperature (907° C. at atmospheric pressure) and the obtained liquid zinc is optionally introduced into syngas producing unit 400 as described above.

After substantially all the zinc is condensed, gaseous CO enters into combustor 625, where the CO is oxidized with air, to produce CO₂ and heat. Optionally, the heat produced in combustor 625 is transferred to reactor 605 with heat exchanger 630 to compensate for heat losses due to the endothermic reaction between zinc oxide and carbon. Heat exchanger 630 optionally heats reactor 605 from outside and keeps reactor 605 at a temperature somewhat above the melting temperature of zinc, for instance, about 1000° C. Optionally, carbon reservoir 612 receives carbon from a pyrolysis chamber (635), having an inlet for carbonaceous material. Optionally, pyrolysis chamber has also an air inlet (not shown), allowing partial oxidation of the carbonaceous material. The carbonaceous material optionally comprises vegetation, switchgrass (Panicum virgatum), hay, forage, and waste residues from processing plant materials, for instance, sugarcane bagasse vinasse, and/or sawdust. The carbonaceous gasified in chamber 635 to obtain combustible gases and carbon. The carbon exits into reservoir 612, from where it arrives at reactor 605. Optionally, the carbon is delivered from combustor 635 directly to reactor 605, and reservoir 612 is omitted. The gases are optionally oxidized (for example, in oxidizer 625), and heat obtained in this oxidation is used for heating, from outside, reactor 605 and/or pyrolysis chamber 635.

Optionally, the gases that exit pyrolysis chamber 635 enter combustor 625, where they are oxidized. Heat Obtained in this oxidation is optionally supplied to reactor 605, to maintain the zinc in reactor 605 boiling, and/or to pyrolysis chamber 635, to supply heat for the pyrolysis of the carbonaceous material. In another exemplary embodiment, carbonaceous material, rather than carbon, is introduced to reactor 605. The carbonaceous material reduces the zinc oxide to provide zinc and various gases, comprising, for example, gaseous hydrocarbons, the exact composition of which depends on the composition of the carbonaceous material used for reducing the oxide. These, gases are optionally combusted in combustor 625 to heat reactor 605 and keep the zinc at boiling point. In this option, pyrolysis chamber 635 may be omitted, and reservoir 612 is a reservoir of carbonaceous material. The carbonaceous mated al optionally comprises vegetation, switehgrass (Panicum virgatum), hay, forage, and waste residues from processing plant materials, for instance, sugarcane bagasse, vinasse bagass, corn-straw, other kinds of straw, and/or sawdust.

Optionally, reactor 605 receives both carbonaceous materials and carbon particles.

Exemplary Method of Producing Fuel from Sugarcane

FIG. 4 is a flowchart describing actions taken in a method 900 of producing liquid fuel from sugarcane according to an embodiment of the invention.

Sugarcane comprises sugar juice; bagasse; and leaves and stems. The method of FIG. 4 can make uses of all these components at actions 910, 920, 930 respectively.

At 910, ethanol is produced from the sugar juice by microbiologic conversion, optionally, in methods known in the art.

At 920, other portions of the sugarcane are microbiologically converted to biogas. The other portions may comprise, for example, bagasse and portions of the sugar juice that are not fermented to ethanol, for example, vinasse. In some embodiments, the non-fermented sugar portions are completely non-fermentable to ethanol. In other embodiments, the non-fermented sugar portions comprise fermentable and non-fermentable sugar-juice portions.

At 930, the remaining of the sugarcane, for instance, leaves and stems, are combusted; and the heat of their combustion is used for generating electrical power.

Going hack to action 920, the biogas obtained is reacted, at action 922, with water and metal to form syngas. As a by-product, metal oxide is formed.

At 924, the metal is regenerated from the metal oxide. Optionally, the regeneration comprises electrolysis, or other electric power consuming processes. Optionally, some or all of the electric power utilized to regenerate the metal is obtained from combusting the leaves and stems, at action 930. Metal regenerated at action 924 is optionally used for reaction with the biogas, in action 922.

At 926, the syngas produced at 922 is reacted to obtain liquid fuel, for instance, methanol.

It should be noted that the method of FIG. 4 may he practiced without fermenting sugar to ethanol. The sugar may be marketed for eating, fermented to methane, or be used for any other purpose. Nevertheless, as there are many facilities that already use sugar fermentation to ethanol, the method as depicted in FIG. 4 is useful for increasing the fuel output of such facilities, without decreasing the ethanol production, if such decreasing is not desirable.

General Comments

It is appreciated that certain features of the invention, which are, for clarity, described in the context of separate embodiments, may also be provided in combination in a single embodiment. Conversely, various features of the invention, which are, for brevity, described in the context of a single embodiment, ma also be provided separately or in any suitable subcombination.

Although the invention has been described in conjunction with specific embodiments thereof, it is evident that many alternatives, modifications and variations of these embodiments will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

The terms “recycle” and “regenerate” are used in the present description and claims interchangeably.

The terms “sprinkle” and “spray” are used in the present description and claims interchangeably.

What is claimed is:

1. A method comprising:
microbiologically converting biological material to form biogas comprising methane and carbon dioxide,
introducing zinc, water and the biogas into a reaction chamber, to concurrently:
react the zinc, water and the biogas to produce syngas and zinc oxide, and
regenerate zinc from the zinc oxide in the reaction chamber, by allowing the zinc oxide to react with the methane, and
producing a carbonaceous fuel from the syngas.

2. The method of claim 1, wherein the microbial conversion is carried out by cutting vegetation, silaging the cut vegetation and fermenting the silaged vegetation.

3. The method of claim 1, wherein the zinc is introduced as liquid zinc.

4. The method of claim 1, wherein the zinc is introduced as zinc vapor.

5. The method of claim 4, wherein the zinc reacts with the water and biogas as zinc vapor and is regenerated from the zinc oxide as liquid zinc.

6. The method of claim 5, wherein the biological material comprises sugarcane bagasse and/or portions of sugarcane that are not fermentable to ethanol, and further comprising:
gasifying the biological material to generate combustible gases, and boiling the liquid zinc with heat obtained from oxidizing the combustible gases.

7. The method of claim 5, further comprising:
producing electrical power by combusting cut vegetation, and
boiling the liquid zinc using the produced electrical power.

8. The method according to claim 1, further comprising:
forming alcohol from the produced syngas,
microbiologically converting vegetation to form carboxylic acid, and
reacting the alcohol with the carboxylic acid to obtain an ester.

9. The method according to claim 8, wherein the carboxylic acid comprises at least one of succinic acid and butyric acid.

10. A system comprising
a fermentation unit, having an inlet for biological material and an outlet for biogas, and arranged to microbiologically convert the biological material to form biogas comprising methane and carbon dioxide,
a syngas producing unit arranged to receive the biogas from the fermentation unit and introduce zinc, water and the biogas into a reaction chamber, to concurrently react the zinc, water and the biogas to produce syngas and zinc oxide, and regenerate zinc from the zinc oxide in the reaction chamber, by allowing the zinc oxide to react with the methane, and
a fuel producing unit arrange to receive the syngas from the syngas production unit and produce a carbonaceous fuel therefrom.

11. The system of claim 10, wherein the introduced zinc is gaseous, and the syngas producing unit comprises a reactor arranged to boil liquid regenerated zinc.

12. The system of claim 11, wherein the reactor comprises a heat exchanger for boiling the regenerated zinc.

13. The system of claim 10, wherein the introduced zinc is liquid.
